(19) United States
(12) Patent Application Publication
(10) Pub. No.: US 2023/0015369 A1
(43) Pub. Date: Jan. 19, 2023

(54) SYSTEM AND METHOD FOR CONTROLLING AN ULTRASONIC SURGICAL SYSTEM

(71) Applicant: Covidien LP, Mansfield, MA (US)

(72) Inventors: Jing Zhao, Superior, CO (US); Christopher T. Brown, Bolder, CO (US); Christopher Tschudy, Arvada, CO (US); Anjali Dhiman, Commerce City, CO (US); Robert H. Wham, Boulder, CO (US); Kelly Goodman, Boulder, CO (US); David Van Tol, Boulder, CO (US); Kristen Bradley, Westminster, CO (US)

(21) Appl. No.: 17/785,494

(22) PCT Filed: Jan. 4, 2021
(86) PCT No.: PCT/US2021/012062

§ 371 Date: Jun. 15, 2022

Related U.S. Application Data
(60) Provisional application No. 62/961,818, filed on Jan. 16, 2020.

(51) Int. Cl. A61B 17/32

(52) U.S. Cl. CPC A61B 17/320068; A61B 2017/320074; A61B 2018/0063

(57) ABSTRACT

A computer implemented method for controlling an ultrasonic surgical system includes activating an ultrasonic surgical system including an ultrasonic generator, an ultrasonic transducer, and an ultrasonic blade. The method further includes collecting data from the ultrasonic surgical system, communicating the data to a machine learning algorithm, determining the vessel size based on the data, using the machine learning algorithm, communicating the determined vessel size to a computing device associated with the ultrasonic generator, and controlling the activated ultrasonic surgical system in accordance with the vessel size. The data may include an electrical parameter associated with the activated ultrasonic surgical system. When the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade.

### CROSS-REFERENCE TO RELATED APPLICATIONS

The present application is a U.S. National Stage Application filed under 35 U.S.C. § 371(a) claiming the benefit of and priority to International Patent Application No. PCT/US2021/012062, filed Jan. 4, 2021, which claims the benefit of and priority to U.S. Provisional Patent Application Ser. No. 62/961,818, filed Jan. 16, 2020, the entire disclosures of each of which being incorporated by reference herein.

### BACKGROUND

### Technical Field

The disclosure relates to electrosurgical procedures and, more particularly, to systems and methods for controlling an ultrasonic surgical system.

### Background of Related Art

Surgical instruments are utilized to perform various functions on tissue structures. An example of such a surgical instrument is an ultrasonic surgical instrument that utilizes ultrasonic energy, i.e., ultrasonic vibrations, to treat tissue. More specifically, a typical ultrasonic surgical instrument utilizes mechanical vibration energy transmitted at ultrasonic frequencies to coagulate, cauterize, fuse, seal, cut, desiccate, fulgurate, or otherwise treat tissue.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

In accordance with aspects of the disclosure, a computer-implemented method for controlling a surgical system is provided. The computer-implemented method includes activating an ultrasonic surgical system including an ultrasonic generator, an ultrasonic transducer, and an ultrasonic blade. The method further includes collecting data from the ultrasonic surgical system, including an electrical parameter associated with the activated ultrasonic surgical system. The method additionally includes communicating the data to a machine learning algorithm, determining the vessel size based on the data using the machine learning algorithm, communicating the determined vessel size to a computing device associated with the ultrasonic generator, and controlling the activated ultrasonic surgical system in accordance with the vessel size. When the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade.

In an aspect of the present disclosure, controlling the activated ultrasonic surgical system includes determining when to stop generating, by the ultrasonic generator, the drive signal, wherein the drive signal is for sealing the vessel. A second drive signal is generated, by the ultrasonic generator, for cutting the vessel, based on the determining.

In another aspect of the present disclosure, the data from the ultrasonic surgical system may include a voltage, a current, a frequency, a velocity, a TransV, a TransVPhase, MFB, Z_ph, or df/dt.

In an aspect of the present disclosure, a machine learning algorithm may include a neural network.

In yet another aspect of the present disclosure, the neural network may include a temporal convolutional network or a feed-forward network.

In a further aspect of the present disclosure, the computer-implemented method may further include training the neural network by accessing ultrasonic surgical system data or identifying patterns in data.

In an aspect of the present disclosure, the computer-implemented method may further include training the neural network to use training data, which may include: a voltage, a current, a frequency, a velocity, a TransV, a TransVPhase, MFB, Z_ph, or df/dt.

In a further aspect of the present disclosure, training the neural network may include supervised training, unsupervised training, or reinforcement learning.

In accordance with aspects of the disclosure, a system for controlling an ultrasonic surgical procedure is presented. The system includes an ultrasonic generator, an ultrasonic transducer, an ultrasonic blade, a processor, and a memory coupled to the processor. When the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade. The memory coupled to the processor includes instructions, which when executed by the processor, cause the system to: collect data from the ultrasonic surgical system, communicate the data to a machine learning algorithm, determine by the machine learning algorithm the vessel size based on the data, communicate the determined vessel size to a computing device, and control the activated ultrasonic surgical system in accordance with the vessel size. The data includes an electrical parameter associated with the activated ultrasonic surgical system. The computing device is associated with the ultrasonic generator.

In a further aspect of the present disclosure, controlling the activate ultrasonic surgical system may include: determining when to stop generating, by the ultrasonic generator, a first drive signal for sealing the vessel, and generating, by the ultrasonic generator, a second drive signal for a cutting the vessel, based on the determining.

In yet a further aspect of the present disclosure, collecting the data from the ultrasonic surgical system may include measuring a voltage, a current, a frequency, a velocity, a TransV, a TransVPhase, MFB, Z_ph, or df/dt.

In yet another aspect of the present disclosure, a machine learning program may include a neural network.

In a further aspect of the present disclosure, the neural network may include a temporal convolutional network or a feed-forward network.

In yet a further aspect of the present disclosure, the instructions, when executed by the processor, may further cause the system to train the neural network by accessing ultrasonic surgical system data or identifying patterns in data.

In yet another aspect of the present disclosure, the instructions, when executed by the processor, may further cause the system to train the neural network to use training data which may include: a voltage, a current, a frequency, a velocity, a TransV, a TransVPhase, MFB, Z_ph, or df/dt.

In a further aspect of the present disclosure, the training of the neural network may include a supervised, unsupervised training, or reinforcement learning.

In accordance with aspects of the disclosure, a non-transitory storage medium that stores a program, causing a computer to execute a method is presented. The method includes activating an ultrasonic surgical system. The ultrasonic surgical system includes an ultrasonic generator, an ultrasonic transducer, and an ultrasonic blade. The method further includes collecting data from the ultrasonic surgical system, communicating the data to a machine learning algorithm, determining the vessel size based on the data, using the machine learning algorithm, communicating the determined vessel size to a computing device associated with the ultrasonic generator, and controlling the activated ultrasonic surgical system in accordance with the vessel size. When the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade. The data includes an electrical parameter associated with the activated ultrasonic surgical system.

In an aspect of the present disclosure, controlling the activated ultrasonic surgical system includes determining when to stop generating, by the ultrasonic generator, the drive signal, wherein the drive signal is for sealing the vessel. A second drive signal is generated, by the ultrasonic generator, for cutting the vessel, based on the determining.

In another aspect of the present disclosure, the data from the ultrasonic surgical system may include a voltage, a current, a frequency, a velocity, a TransV, a TransVPhase, MFB, Z_ph, or df/dt.

In an aspect of the present disclosure, a machine learning algorithm may include a neural network.

In yet another aspect of the present disclosure, the neural network may include a temporal convolutional network or a feed-forward network.

### BRIEF DESCRIPTION OF THE DRAWINGS

Various aspects and features of the disclosure are described herein with reference to the drawings wherein:

FIG. 1 is a perspective view of an ultrasonic surgical system including an ultrasonic surgical instrument having an on-board generator, power source, and transducer provided in accordance with the disclosure;

FIG. 2 is a block diagram of the generator of the surgical system of FIG. 1 in accordance with the disclosure;

FIG. 3 is a block diagram of a controller provided in accordance with the disclosure and configured for use with the surgical system of FIG. 1 in accordance with the disclosure;

FIG. 4 is a logic diagram of a machine learning algorithm in accordance with the disclosure;

FIG. 5 is a diagram of a data record in accordance with the disclosure;

FIG. 6 is an illustration of an energy profile of the generator of the surgical system of FIG. 1 in accordance with the disclosure;

FIG. 7 is an illustration of activation time vs. vessel diameter for a surgical system without training in accordance with the disclosure;

FIG. 8 is a flowchart of a method for estimating vessel diameter in accordance with the disclosure; and

FIG. 9 is an illustration of actual vs. predicted vessel diameter for a surgical system with training in accordance with the disclosure.

### DETAILED DESCRIPTION

Tissue sealing involves heating tissue to liquefy the collagen and elastin in the tissue so that it reforms into a fused mass with significantly-reduced demarcation between the opposing tissue structures. To achieve a tissue seal without causing unwanted damage to tissue at the surgical site or collateral damage to adjacent tissue, it is necessary to control the application of energy to tissue, thereby controlling the temperature of tissue during the sealing process.

With respect to utilizing vessel size information in real-time in order to control the application of energy to tissue to achieve a tissue seal, it would be desirable to determine vessel size during the initial stages of the tissue sealing process to improve seal quality based on measurement data. As detailed below, this may be accomplished by utilizing data available from the surgical system and running a machine learning algorithm to estimate vessel size based upon that data. The estimated vessel size may then be fed back to a controller for use in controlling the application of energy to tissue in accordance therewith. The vessel size may include, but is not limited to vessel diameter, vessel mass, tissue surface area, and/or tissue mass.

The systems and methods herein are not limited to estimating vessel diameter. In various embodiments, the systems and methods may estimate vessel mass (or tissue mass) and then utilize vessel mass (or tissue mass) to detect and adjust for tissue types. For example, the tissue types may include both vascular and non-vascular, arteries vs. veins, etc. In various embodiments, the system may adjust for thin and thick tissue, small and large vessels (veins, arteries), pulmonary vasculature, etc.

The systems and methods of the disclosure detailed below may be incorporated into any type of surgical system for treating tissue such as, for example, the ultrasonic surgical systems detailed hereinbelow. For purposes of illustration and in no way limiting the scope of the appended claims, the systems and methods for estimating vessel diameter for use in controlling the application of energy to tissue are described in the disclosure in the context of ultrasonic surgical systems.

The terms “artificial intelligence,” “data models,” or “machine learning” may include, but are not limited to, neural networks, convolutional neural networks (CNN), recurrent neural networks (RNN), generative adversarial networks (GAN), Bayesian Regression, Naive Bayes, nearest neighbors, least squares, means, and support vector regression, among other data science and artificial science techniques.

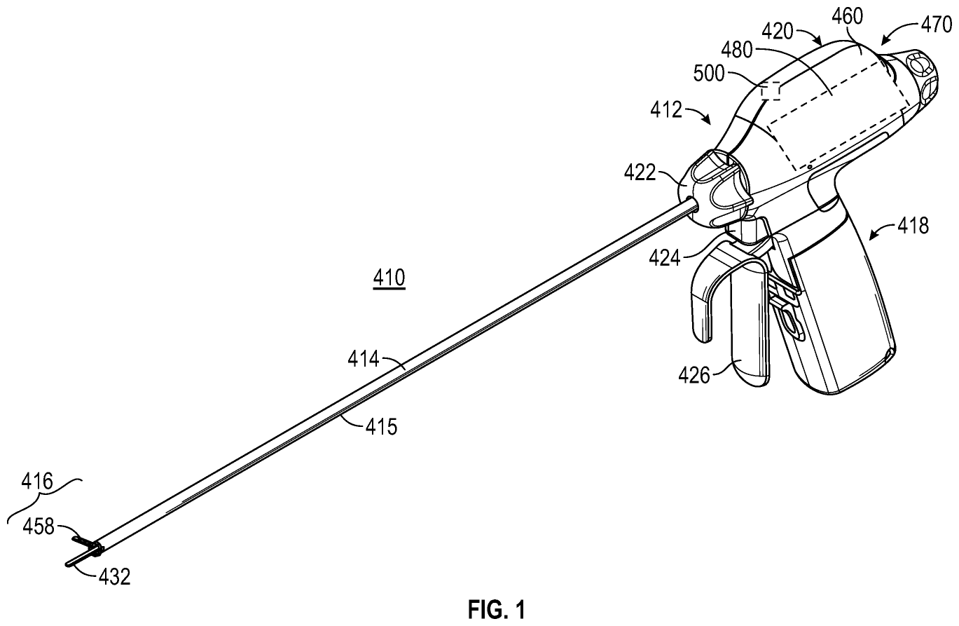

The term “application” may include a computer program designed to perform particular functions, tasks, or activities for the benefit of a user. Application may refer to, for example, software running locally or remotely, as a standalone program or in a web browser, or other software which would be understood by one skilled in the art to be an application. An application may run on a controller, e.g., controller 500 (FIG. 1), or on a user device, including, for example, a mobile device, an IoT device, or a server system.

Referring now to FIG. 1, an ultrasonic surgical system provided in accordance with the disclosure includes an ultrasonic surgical instrument 410 that generally includes a handle assembly 412, an elongated body portion 414, and a tool assembly 416. Tool assembly 416 includes a blade 432 and a clamp member 458. Handle assembly 412 supports a battery assembly 418 and an ultrasonic transducer and generator assembly (“TAG”) 420 including an ultrasonic generator 470 and an ultrasonic transducer 480, although generator 470 and ultrasonic transducer 80 may alternatively be separate components. Handle assembly 412 further includes a rotatable nozzle 422, an activation button 424, and a clamp trigger 426. Battery assembly 418 and TAG 420 are each releasably secured to handle assembly 412 and are removable therefrom to facilitate disposal of the entire device, with the exception of battery assembly 418 and TAG 420. However, it is contemplated that any or all of the components of ultrasonic surgical instrument 410 be configured as disposable single-use components or sterilizable multi-use components. Further, ultrasonic surgical instrument 410 may be configured to connect to a remote generator and/or power source, rather than having such components on-board.

With continued reference to FIG. 1, elongated body portion 414 includes an outer shaft assembly 415 and waveguide (not shown) which extends distally from handle assembly 412 through outer shaft assembly 415 to tool assembly 416. A distal end of the waveguide defines blade 432. A proximal end of the waveguide is configured to engage ultrasonic transducer 480 of TAG 420. The waveguide and outer shaft assembly 415 are rotatably coupled to rotatable nozzle 422 such that rotation of nozzle 422 effects corresponding rotation of the outer shaft assembly 415 and the waveguide. The outer shaft assembly 415 includes a support tube and an actuator tube which are disposed about one another in either configuration.

The actuator tube of outer shaft assembly 415 is configured to move relative to the support tube of outer shaft assembly 415 to enable pivoting of clamp member 458 between an open position, wherein clamp member 458 is spaced from blade 432, and a closed position, wherein clamp member 458 is approximated relative to blade 432. Clamp member 458 is moved between the open and closed positions in response to actuation of clamp trigger 426.

Continuing with reference to FIG. 1, activation button 424 is supported on handle assembly 412. When activation button 424 is activated in an appropriate manner, an underlying two-mode switch assembly is activated to effect communication between battery assembly 418 and TAG 420 in either a “LOW” power mode or a “HIGH” power mode, depending upon the manner of activation button 424.

TAG 420, as noted above, includes generator 470 and ultrasonic transducer 480. Generator 470 includes an outer housing 460 that houses a TAG microcontroller 500 having a memory. TAG 420 supports the ultrasonic transducer 480 thereon. The ultrasonic transducer 480 may include a piezoelectric stack and defines a forwardly extending horn configured to engage the proximal end of the waveguide. A series of contacts (not explicitly shown) associated with TAG 420 enable communication of power and/or control signals between TAG 420, battery assembly 418, and the two-mode switch assembly, although contactless communication therebetween is also contemplated.

In general, in use, when battery assembly 418 and TAG 420 are attached to handle assembly 412 and the waveguide, respectively, and ultrasonic surgical instrument 410 is activated, battery assembly 418 provide power to generator 470 of TAG 420 which, in turn, uses this power to apply an AC signal to the ultrasonic transducer 480 of TAG 420. The ultrasonic transducer 480, in turn, converts the AC signal into high-frequency mechanical motion. This high-frequency mechanical motion produced by the ultrasonic transducer 480 is transmitted along the waveguide to the blade 432 for application of such ultrasonic energy to tissue adjacent to or clamped between blade 432 and clamp member 458 to treat tissue.

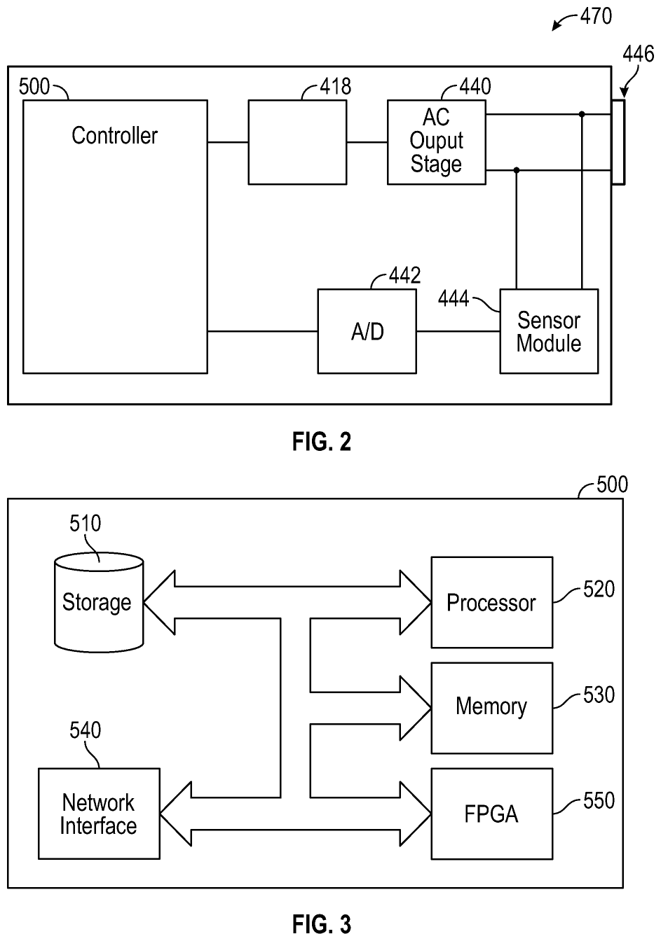

Referring now to FIG. 2, a block diagram of the generator 470 of the surgical system of FIG. 1 in accordance with the disclosure is shown. In various embodiments, the generator 470 may include a sensor module 444, which includes a plurality of sensors, e.g., a current sensor, and a voltage sensor. Various components of the generator 470, namely, the AC output stage 440 and the AC current and voltage sensors of sensor module 444 may be disposed on a printed circuit board (PCB). The AC current sensor of sensor module 444 may be coupled to an active terminal on the ultrasonic transducer 480 (FIG. 1) and provides measurements of the AC current supplied by the AC output stage 440. In embodiments the AC current sensor of sensor module 444 may be coupled to the return terminal on the ultrasonic transducer 480 (FIG. 1). The AC voltage sensor of sensor module 444 is coupled to the active and return terminals on the ultrasonic transducer 480 (FIG. 1) and provides measurements of the AC voltage supplied by the AC output stage 440.

The AC current and voltage sensors of the sensor module 444 sense and provide the sensed AC voltage and current signals, respectively, to the controller 500 of generator 470, which then may adjust output of battery assembly 418 and/or the AC output stage 440 in response to the sensed AC voltage and current signals. Controller 500 is described in greater detail hereinbelow (see FIG. 3).

The sensed voltage and current from sensor module 444 are fed to analog-to-digital converters (ADCs) 442. The ADCs 442 sample the sensed voltage and current to obtain digital samples of the voltage and current of the AC output stage 440. The digital samples are processed by the controller 500 and used to generate a control signal to control the DC/AC inverter of the AC output stage 440. The ADCs 442 communicate the digital samples to the controller 500 for further processing.

In various embodiments, the controller 500 may collect data relating to the generator 470 during use, including voltage, current, power, frequency, velocity, or any parameters derived from these signals such as AC voltage applied to the transducer (TransV), AC current applied to the transducer (Transl), phase angle between TransV and the phase reference signal (TransVPhase), Motional feedback bridge (MFB), impedance phase (Z_ph), or df/dt. For example, with respect to the ultrasonic surgical system of FIG. 1, the ultrasonic surgical system may be used to apply ultrasonic energy to tissue to treat tissue. More specifically, with additional reference to FIG. 1, tissue (not shown) is clamped between blade 432 and clamp member 458 and an AC signal is applied to ultrasonic transducer 480 of TAG 420, which in turn, converts the AC signal to high-frequency mechanical motion. This high-frequency mechanical motion produced by the ultrasonic transducer 480 is transmitted along the waveguide to the blade 432, where the high-frequency motion is used to treat the tissue clamped between the blade 432 and clamp member 458.

During such tissue treatment, the sensor circuitry, e.g., sensor module 444, of the generator 470 may sense parameters of the tissue, system, and/or energy (ultrasonic energy) such as, for example, voltage, current, frequency, velocity, TransV, TransVPhase, MFB, Z_ph, and/or df/dt. This may occur as a snapshot or over a time interval and may be determined at the beginning of tissue treatment, e.g., at or within 250 ms of initiation of tissue treatment. The sensed data may include, for example, time that the power is applied to ultrasonic transducer 480. The sensor module 444 may measure data from the system, for example, the voltage and/or a current of the drive signal delivered to the ultrasonic transducer 480. This sensed data obtained by the sensor circuitry is relayed to the controller 500 (via the ADC's 442, in embodiments) for further processing, as detailed below.

In various embodiments, the controller 500 uses the stored settings and the parameters as training data for a machine learning algorithm. In various embodiments, training the machine learning algorithm may be performed by a computing device outside of the generator 470, and the resulting algorithm may be communicated to the controller 500 of generator 470. In various embodiments, the controller 500 communicates the determined vessel diameter that was output from the machine learning algorithm to a computing device, e.g., of controller 500, for use in formulating, e.g., switching, confirming, modifying, generating, etc., a tissue sealing algorithm. In various embodiments, the controller 500 adjusts, on the generator 470, an algorithm that controls a sealing cycle (by adjusting the drive signal from generator 470 to ultrasonic transducer 480), based on the output of the machine learning algorithm. In various embodiments, the machine learning algorithm network may use supervised learning, unsupervised learning, or reinforcement learning. In various embodiments, the neural network may include a temporal convolutional network, with one or more fully connected layers, or a feed forward network. In various embodiments, the training may happen on a separate system. In various embodiments, the controller 500 may use the stored settings and the sensed parameters for a machine learning algorithm to infer the vessel diameter.

Referring to FIG. 3, the controller 500 is shown. The controller 500 includes a processor 520 connected to a computer-readable storage medium or a memory 530 which may be a volatile type memory, e.g., RAM, or a non-volatile type memory, e.g., flash media, disk media, etc. In various embodiments, the processor 520 may be another type of processor such as, without limitation, a digital signal processor, a microprocessor, an ASIC, a graphics processing unit (GPU), field-programmable gate array (FPGA), or a central processing unit (CPU). In various embodiments, network inference may also be accomplished in systems that may have weights implemented as memistors, chemically, or other inference calculations, as opposed to processors.

In various embodiments, the memory 530 can be random access memory, read-only memory, magnetic disk memory, solid state memory, optical disc memory, and/or another type of memory. In various embodiments, the memory 530 can be separate from the controller 500 and can communicate with the processor 520 through communication buses of a circuit board and/or through communication cables such as serial ATA cables or other types of cables. The memory 530 includes computer-readable instructions that are executable by the processor 520 to operate the controller 500. In various embodiments, the controller 500 may include a network interface 540 to communicate with other computers or a server. In embodiments, a storage device 510 may be used for storing data. In various embodiments, the controller 500 may include one or more FPGAs 550. The FPGA 550 may be used for executing various machine learning algorithms such as those provided in accordance with the disclosure, as detailed below.

The memory 530 stores suitable instructions, to be executed by the processor 520, for receiving the sensed data, e.g., sensed data from sensor module 444 via ADCs 442 (see FIG. 2), accessing storage device 510 of the controller 500, determining one or more tissue parameters, e.g., vessel diameter, based upon the sensed data and information stored in storage device 510, and providing feedback based upon the determined tissue parameter(s). Although illustrated as part of generator 470, it is also contemplated that controller 500 be remote from generator 470, e.g., on a remote server, and accessible by generator 470via a wired or wireless connection. In embodiments where controller 500 is remote, it is contemplated that controller 500 may be accessible by and connected to multiple generators 470.

Storage device 510 of controller 500 stores one or more machine learning algorithms and/or models, configured to estimate one or more tissue parameters, e.g., vessel diameter, vessel mass, and/or tissue mass, based upon the sensed data received from sensory circuitry, e.g., from sensor module 444 via ADCs 442 (see FIG. 2). The machine learning algorithm(s) may be trained on and learn from experimental data and/or data from previous procedures initially input into the one or more machine learning applications in order to enable the machine learning application(s) to predict the vessel diameter (or vessel mass) based upon such data. Such data may include voltage (e.g., a transducer voltage), current (e.g., a transducer current), frequency (e.g., an activation frequency), velocity (e.g., a blade velocity), TransV, TransVPhase, MFB, Z_ph, df/dt, a change in activation over time, and/or any other suitable data.

Referring generally to FIG. 2, machine learning algorithms are advantageous for use in predicting the vessel diameter (vessel mass and/or tissue mass) at least in that complex sensor components and pre-defined categorization rules and/or algorithms are not required. Rather, machine learning algorithms utilize the initially input data, e.g., the previous procedure data and/or experimental data, to determine statistical features and/or correlations that enable the prediction of vessel diameter (vessel mass and/or tissue mass) by analyzing data therefrom. Thus, with the one or more machine learning algorithms having been trained as detailed above, such can be used to determine the vessel diameter (or vessel and/or tissue mass) of tissue being treated using ultrasonic surgical instrument 410. More specifically, processor 520 of controller 500 is configured, in response to receiving sensed data from sensory circuitry, e.g., from sensor module 444 via ADCs 442, to input the sensed data into the machine learning algorithm(s) stored in storage device 510 in order to determine the vessel diameter of the tissue being treated. Although described with respect to an ultrasonic surgical system, the aspects and features of controller 500 and the machine learning algorithms configured for use therewith are equally applicable for use with other suitable surgical systems, e.g., an electrosurgical system.

Once the vessel diameter is determined by the controller 500, depending upon the vessel diameter, settings, user input, etc., controller 500 may for example, output an alert and/or warning to user interface, implement, switch, or modify a particular tissue sealing algorithm based upon which the battery cells of battery assembly 418 and AC output stage 440 provide energy to the ultrasonic transducer 480, modify the energy provided to the ultrasonic transducer 480, and/or inhibit further energy delivery to the ultrasonic transducer 480.

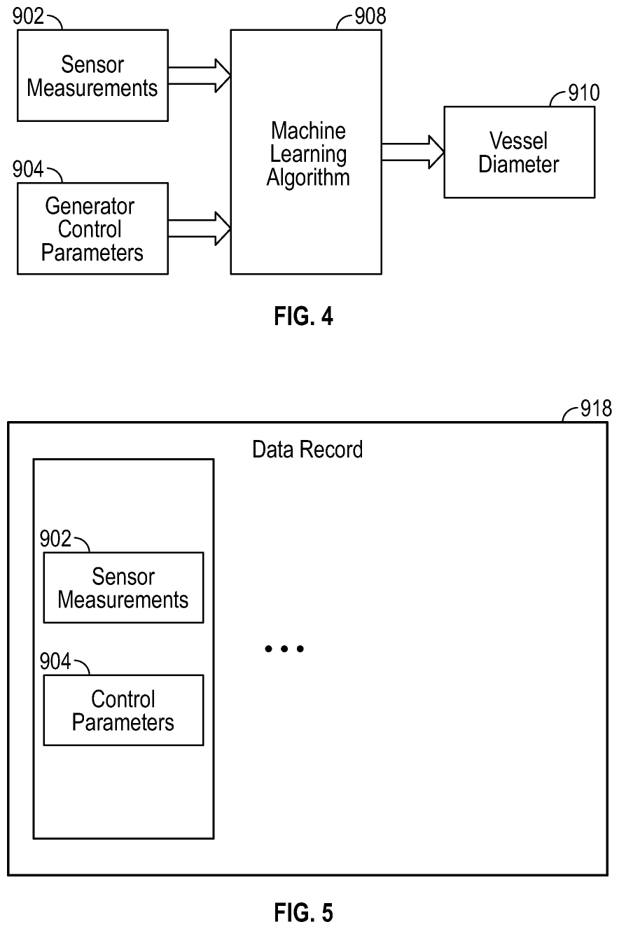

With reference to FIG. 4, a logic diagram of a machine learning algorithm 908 is shown in accordance with the disclosure. Training of the machine learning algorithm 908 may include using sensor measurements 902 and generator control parameters 904 as inputs to the machine learning algorithm 908. The machine learning algorithm 908 outputs a prediction of a vessel diameter 910 (vessel mass, and/or tissue mass). A data record 918 (FIG. 5) may include multiple sensor measurements 902, and/or associated generator control parameters 904 that are used to train the machine learning algorithm 908. In various embodiments, training may include accessing ultrasonic surgical system data or identifying patterns in data.

In various embodiments, the generator control parameters 904 that correlate with particular sensor measurements 902 of are used as inputs to the machine learning algorithm during training. In various embodiments, the generator control parameters 904 may include, for example, time, slope, or other generator 470 parameters. In various embodiments, the controller 500 may communicate to a remote server, for example, the stored adjusted control parameters, text data, and/or the output of the machine learning algorithm.

In various embodiments, the outputs of the neural network may be used as training data for supervised learning, unsupervised learning, or reinforcement learning. It is contemplated that the training may be performed on a separate system, for example, GPU workstations, High Performing Computer Clusters, etc., and the trained network would then be deployed in the ultrasonic surgical system. In various embodiments, the controller 500 outputs, from the machine learning algorithm, a prediction of the vessel diameter (vessel mass and/or tissue mass) based on the inputs.

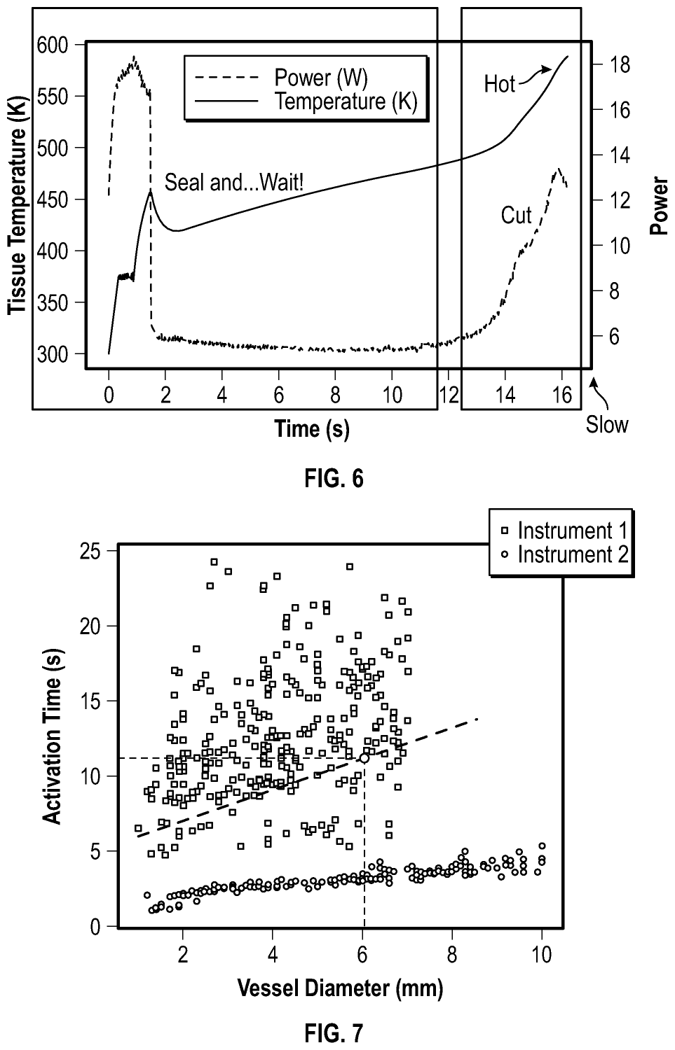

Referring now to FIG. 6, an illustration of an energy profile of the generator of the surgical system of FIG. 1 in accordance with the disclosure is shown. For example, the generator provides a suitable drive signal to the ultrasonic transducer to produce ultrasonic energy that is applied to the tissue. Initially, the drive signal is applied to achieve a tissue seal, e.g., according to a tissue sealing algorithm. As the energy is applied to the tissue, the tissue temperature increases. After a period of time has elapsed and a tissue seal has completely formed, the generator then switches to apply the drive signal to cut tissue, e.g., according to a tissue cutting algorithm. Depending upon the vessel diameter of the tissue being treated, parameters associated with sealing and cutting the tissue may vary. For example, the sealing drive signal, the cutting drive signal, the duration of application of the sealing and/or cutting drive signals, etc. may be different depending upon the vessel diameter of the tissue being treated. It is important to ensure that a vessel is sufficiently sealed prior to cutting the vessel. On the other hand, it is beneficial to reduce the overall time required to seal and cut tissue.

Referring now to Instrument 2 of FIG. 7, an illustration of activation time vs. vessel diameter for a surgical system without the knowledge of vessel diameter in accordance with the disclosure is shown. In various embodiments, a minimum activation time required to achieve a satisfactory seal (e.g., a seal having a minimum burst pressure strength) may be determined empirically for a vessel with known diameter such as Instrument 1. In various embodiments, the machine learning algorithm may be used to predict vessel diameter (vessel mass and/or tissue mass) early (e.g., within first 5 seconds of activation) to determine when to stop the sealing drive signal and transition to the cut drive signal. Therefore, the total device activation time as a function of vessel diameter may be approximated by the dash line in FIG. 7A including safety margins.

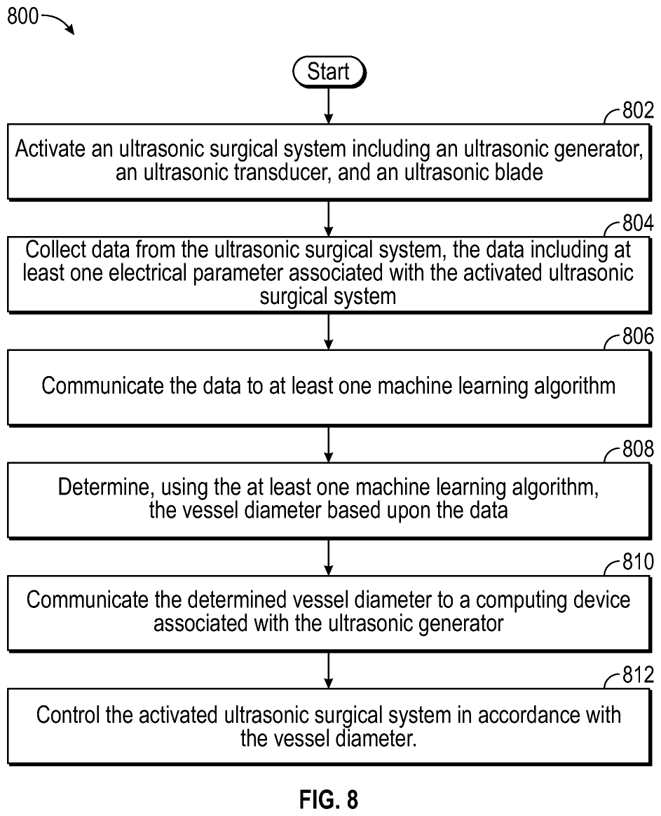

Referring now to FIG. 8, there is shown a flow diagram of a computer-implemented method 800 for estimating a vessel diameter. Persons skilled in the art will appreciate that one or more operations of the method 800 may be performed in a different order, repeated, and/or omitted without departing from the scope of the disclosure. In various embodiments, the illustrated method 800 can operate in the controller 500 (FIG. 3), in a remote device, or in another server or system. In various embodiments, some or all of the operations in the illustrated method 800 can operate using an ultrasonic surgical system, e.g., instrument 410. Other variations are contemplated to be within the scope of the disclosure. The operations of FIG. 8 will be described with respect to a controller, e.g., controller 500 of generator 470 (FIGS. 2 and 3), but it will be understood that the illustrated operations are applicable to other systems and components thereof as well.

Initially, at step 802, the controller 500 may activate an ultrasonic surgical system. The ultrasonic surgical system includes an ultrasonic generator 470, an ultrasonic transducer 480, and an ultrasonic blade 432. When the ultrasonic surgical system is activated, the ultrasonic generator 470 produces a drive signal to drive the ultrasonic transducer 480 which, in turn, produces ultrasonic energy that is transmitted to the ultrasonic blade 432 for treating a vessel in contact with the ultrasonic blade 432. The vessel defines a vessel diameter.

At step 804, the controller 500 may collect data from the ultrasonic surgical system. In various embodiments, the data includes electrical parameters associated with the activated ultrasonic surgical system. In various embodiments, the controller 500 may collect data relating to the generator 470, for example, voltage, current, frequency, velocity, TransV, TransVPhase, MFB, Z_ph, or df/dt. The data may be collected during an initial stage of activation, e.g., within the first 5 seconds of activation. At step 806, controller 500 may communicate the data to the machine learning algorithm 908 (e.g., a neural network). In various embodiments, the neural network may include a temporal convolutional network or a feed-forward network. In various embodiments, the machine learning algorithm 908 may be trained using data relating to the generator 470, for example, voltage, current, frequency, velocity, TransV, TransVPhase, MFB, Z_ph, or df/dt. In various embodiments, the training may include supervised training, unsupervised training, or reinforcement learning. In various embodiments, the reinforcement learning may include a reward or a punishment.

At step 808, the controller 500 may determine, using the machine learning algorithm 908, the vessel size based upon the data. The vessel size may include, for example, a vessel diameter, a vessel mass, a tissue surface area, and/or a tissue mass. For example, based on the output for the machine learning algorithm 908, the controller 500, may determine that the vessel diameter is approximately 6 mm. At step 810 the controller 500 may communicate the determined vessel diameter to a computing device associated with the ultrasonic generator 470.

At step 812, the controller 500 may control the activated ultrasonic surgical system in accordance with the vessel size. In various embodiments, the controller 500 may determine when to stop generating, by the ultrasonic generator 470, a first drive signal (e.g., a “seal” drive signal) for driving the ultrasonic transducer 480 to seal the vessel. In various embodiments, the controller 500 may generate, by the ultrasonic generator 470, a second drive signal (e.g., a “cut” drive signal) for driving the ultrasonic transducer to cut the vessel, based on the determining. For example, the controller 500 may determine at approximately 13 seconds to stop generating a “seal” drive signal and may then generate a “cut” drive signal.

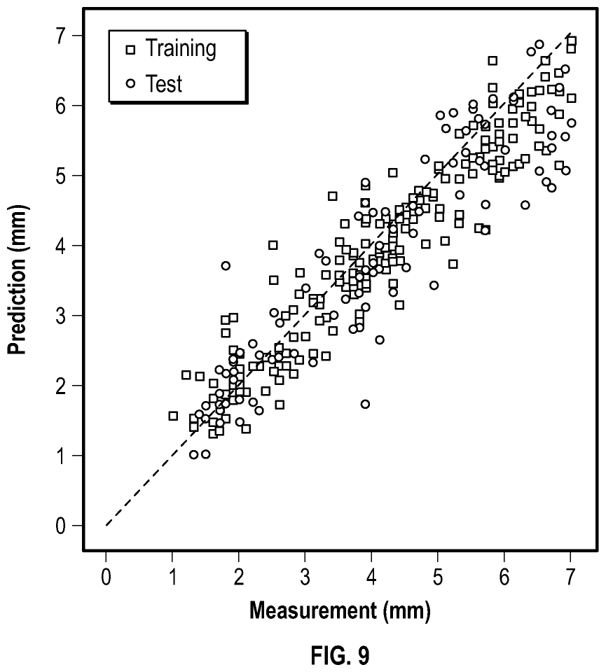

Referring now to FIG. 9, an illustration of actual vs. predicted vessel diameter for a surgical system with training in accordance with the disclosure is shown. In various embodiments, a vessel diameter predicted by the machine learning algorithm 908 may be compared to the actual measured vessel diameter.

From the foregoing and with reference to the various figure drawings, those skilled in the art will appreciate that certain modifications can also be made to the disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A computer-implemented method for controlling an ultrasonic surgical system, the computer-implemented method comprising:
activating an ultrasonic surgical system including an ultrasonic generator, an ultrasonic transducer, and an ultrasonic blade, wherein, when the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade, the vessel defining a vessel size;
collecting data from the ultrasonic surgical system, the data including at least one electrical parameter associated with the activated ultrasonic surgical system;
communicating the data to at least one machine learning algorithm;
determining, using the at least one machine learning algorithm, the vessel size based upon the data;
communicating the determined vessel size to a computing device associated with the ultrasonic generator; and
controlling the activated ultrasonic surgical system in accordance with the vessel size.

2. The computer-implemented method of claim 1, wherein controlling the activated ultrasonic surgical system includes:
determining when to stop generating, by the ultrasonic generator, the drive signal, wherein the drive signal is for sealing the vessel; and
generating, by the ultrasonic generator, a second drive signal for cutting the vessel, based on the determining.

3. The computer-implemented method of claim 1, wherein the data from the ultrasonic surgical system includes at least one of a voltage, a current, a frequency, a velocity, a TransV, a TransVPhase, MFB, Z_ph, or df/dt.

4. The computer-implemented method of claim 1, wherein the at least one machine learning algorithm includes a neural network.

5. The computer-implemented method of claim 4, wherein the neural network includes at least one of a temporal convolutional network or a feed-forward network.

6. The computer-implemented method of claim 4, the method further includes training the neural network using one or more of accessing ultrasonic surgical system data or identifying patterns in data.

7. The computer-implemented method of claim 4, the method further includes training the neural network using training data including at least one of: a voltage, a current, a frequency, a velocity, a TransV, a TransVPhase, MFB, Z_ph, or df/dt.

8. The computer-implemented method of claim 7, wherein the training includes at least one of supervised training, unsupervised training, or reinforcement learning.

9. A system for controlling an ultrasonic surgical procedure, the system comprising:
an ultrasonic generator;
an ultrasonic transducer;
an ultrasonic blade, wherein, when the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade, the vessel defining a vessel size;
one or more processors; and
at least one memory coupled to the one or more processors, the at least one memory having instructions stored thereon which, when executed by the one or more processors, cause the system to:
collect data including at least one electrical parameter associated with the ultrasonic surgical system when activated;
communicate the data to at least one machine learning algorithm;
determine, using the at least one machine learning algorithm, the vessel size based on the data;
communicate the determined vessel size to a computing device associated with the ultrasonic generator; and
control activation of the ultrasonic surgical system in accordance with the vessel size.

10. The system of claim 9, wherein controlling the activated ultrasonic surgical system includes:
determining when to stop generating, by the ultrasonic generator, a first drive signal for sealing the vessel; and
generating, by the ultrasonic generator, a second drive signal for cutting the vessel, based on the determining.

11. The system of claim 9, wherein collecting the data from the ultrasonic surgical system includes measuring at least one of a voltage, a current, a frequency, a velocity, a TransV, a TransVPhase, MFB, Z_ph, or df/dt.

12. The system of claim 9, wherein the at least one machine learning algorithm includes a neural network.

13. The system of claim 12, wherein the neural network includes at least one of a temporal convolutional network or a feed-forward network.

14. The system of claim 12, wherein the instructions, when executed by the one or more processors, further cause the system to train the neural network using one or more of: accessing ultrasonic surgical system data or identifying patterns in data.

15. The system of claim 12, wherein the instructions, when executed by the one or more processors, further cause the system to train the neural network using training data including at least one of: a voltage, a current, a frequency, a velocity, a TransV, a TransVPhase, MFB, Z_ph, or df/dt.

16. The system of claim 15, wherein the training includes at least one of supervised training, unsupervised training, or reinforcement learning.

17. A non-transitory storage medium that stores a program causing a computer to execute a method, the method comprising:
activating an ultrasonic surgical system including an ultrasonic generator, an ultrasonic transducer, and an ultrasonic blade, wherein, when the ultrasonic surgical system is activated, the ultrasonic generator produces a drive signal to drive the ultrasonic transducer which, in turn, produces ultrasonic energy that is transmitted to the ultrasonic blade for treating a vessel in contact with the ultrasonic blade, the vessel defining a vessel size;
collecting data from the ultrasonic surgical system, the data including at least one electrical parameter associated with the activated ultrasonic surgical system;
communicating the data to at least one machine learning algorithm;
determining, using the at least one machine learning algorithm, the vessel size based upon the data;
communicating the determined vessel size to a computing device associated with the ultrasonic generator; and
controlling the activated ultrasonic surgical system in accordance with the vessel size.

18. The computer-implemented method of claim 17, wherein controlling the activated ultrasonic surgical system includes:
determining when to stop generating, by the ultrasonic generator, the drive signal, wherein the drive signal is for sealing the vessel; and
generating, by the ultrasonic generator, a second drive signal for cutting the vessel, based on the determining.

19. The computer-implemented method of claim 17, wherein the data from the ultrasonic surgical system includes at least one of a voltage, a current, a frequency, a velocity, a TransV, a TransVPhase, MFB, Z_ph, or df/dt.

20. The computer-implemented method of claim 17, wherein the at least one machine learning algorithm includes a neural network.
